(19) United States
(12) Patent Application Publication
(10) Pub. No.: US 2022/0403330 A1
(43) Pub. Date: Dec. 22, 2022

(54) COMPOSITIONS CAPABLE OF STIMULATING IMMUNITY TOWARDS TUMOR BLOOD VESSELS

(71) Applicant: Therapeutic Solutions International, Inc., Oceanside, CA (US)

(72) Inventors: Thomas E. Ichim, Oceanside, CA (US); Famela Ramos, Oceanside, CA (US); James Veltmeyer, Oceanside, CA (US); Timothy G. Dixon, Oceanside, CA (US); Feng Lin, Oceanside, CA (US)

(73) Assignee: Therapeutic Solutions International, Inc., Oceanside, CA (US)

(21) Appl. No.: 17/748,189

(22) Filed: May 19, 2022

Related U.S. Application Data
(60) Provisional application No. 63/167,552, filed on Mar. 29, 2021.

(51) Int. Cl. C12N 5/074; A61K 35/12; A61P 9/00

(52) U.S. Cl. CPC C12N 5/0607; A61K 35/12; A61P 9/00; C12N 2501/115

(57) ABSTRACT

Disclosed are novel means, protocols, and compositions of matter for eliciting an immune response against blood vessels supplying neoplastic tissue. In one embodiment pluripotent stem cells are transfected with one or more genes capable of eliciting immunity. In some embodiments such genes are engineered under control of specific promoters to allow for various specificities of activity. In one specific embodiment pluripotent stem cells engineered to endow properties capable of inducing expression of the α-Gal epitope (Galα1,3Galα1,4G1cNAc-R).

### CROSS-REFERENCE TO RELATED APPLICATIONS

This application claims the benefit of priority to U.S. Provisional Application No. 63/167,552, filed Mar. 29, 2021, the entire contents of which is incorporated herein by reference.

### FIELD OF THE INVENTION

The teachings herein pertain to the field of immune modulations, such as to treat cancers and tumors. Specific methods involve the use of cells to stimulate immunity towards tumor blood vessels.

### BACKGROUND

It is widely recognized that a continuous layer of endothelial cells (ECs) lines the heart, arterioles, capillaries, veins, and lymphatics. Scientists have traditionally characterized the endothelium as a dynamic, functioning organ and not a solid structural component, which was the prior view. The endothelium is highly specialized and varies considerably from tissue to tissue and organ to organ. For example, the kidney's glomerulus is a fenestrated capillary tuft that filters blood to form urine whereas the blood—brain barrier endothelium is characterized by junctional proteins that restrict passage of solutes into the central nervous system.

Regardless of its tissue of origin, the endothelium performs several critical functions including regulating the passage of nutrients, oxygen, and other solutes from the bloodstream to the tissues, regulating the flow of blood by maintaining a nonthrombogenic surface, and controlling the trafficking of leukocytes into and out of the tissues.

We recognize that structure of the normal vascular endothelium is hierarchical. Arteries branch to arterioles, which then form thin-walled capillaries. Smooth muscle cells (SMCs) wrap around large vessel endothelium and provide vessel stability and paracrine/juxtacrine cues to the underlying ECs. SMCs also express contractile proteins that regulate vessel diameter. The finer capillaries are surrounded by perivascular cells called pericytes that also provide vessel stability.

In studies that utilized genetic engineering to evoke depletion of PDGFB (a major pericyte growth factor) or its receptor, researchers observed loss of pericytes, vessel leakage, and hemorrhage. It is further understood that all blood vessels have a proteinaceous basement membrane or extracellular matrix (ECM) usually rich in collagens, laminin, and fibronectin. The ECM provides support and stability but can also signal through interactions with integrins expressed on the EC surface.

As we have previously stated, the blood vessels themselves are recognized as dynamic structures. New vessels are formed when they are needed such as during wound healing, whereas old ones are pruned away.

The formation of new blood vessels, otherwise known as neovascularization occurs by three main processes: angiogenesis, vasculogenesis, and intussusception.

In cancer, these same processes are known to occur with the exception that regulatory pathways controlling blood vessel growth, branching, and morphology in tumor vessels are faulty. In addition, some of the same processes regulating blood vessel patterning and growth during development of the embryo reappear in tumor angiogenesis. A fourth process termed arteriogenesis involves an increase in the diameter of preexisting arterioles that remodel and form collaterals, but this process is not well-described in the tumor vasculature and is not discussed here.

The creation of new blood vessels that arise from preexisting ones usually happens by the process of angiogenesis. Angiogenesis is characterized by dissolution of the ECM, multiplication of the endothelial cells and sprouting. Vascular patterning is controlled by gradients of angiogenic factors that guide and unite immature endothelial tip cells.

We know, for example, that the VEGF/D114/notch axis is a key regulator of vessel sprouting. Interestingly, neuronal and vascular systems share common guidance cues.

An intrinsic pathway using local expression gradients of sFLT-1 that directs emerging sprouts away from the parent vessel was recently proposed. After a new vessel is formed, the basement membrane and pericytes add stability to the nascent vascular tree. Angiogenesis is a tightly controlled, self-regulating, reversible process. For example, the formation of new blood vessels has evolved so that products generated during ECM remodeling can inhibit EC proliferation and migration, thus fine-tuning the formation of new vascular structures.

In contrast to angiogenesis, vasculogenesis occurs mainly during development when progenitor cells (angioblasts) committed to the vascular lineage differentiate to form an immature vascular plexus in the embryo.

In the yolk sac, a bi-potent stem cell called the hemangioblast is thought to differentiate to form both hematopoietic cells and ECs. Aggregates of mesodermal cells within the yolk sac form a blood island that gives rise to ECs at the periphery and hematopoietic cells in the center. Shared expression of a number of different markers supports the concept that ECs and hematopoietic cells have a common ancestor; however, unequivocal evidence for the existence of the hemangioblast is still debated. Strong evidence for the generation of hematopoietic cells through hemogenic endothelium was recently presented. Bona fide ECs for postnatal vasculogenesis can be isolated from the peripheral blood of adults, but the origin of these circulating ECs remains elusive. Furthermore, the role of circulating ECs in tumor angiogenesis remains controversial.

An alternative and rapid mechanism for a new vessel to form is through intussusceptive angiogenesis (IA). During IA, the capillary wall forms transvascular tissue pillars and extends into the lumen splitting the vessel on the long axis while maintaining intact circulation. Because the process occurs by reorganization of existing cells, IA allows for the rapid increase in capillary density in the developing embryo and perhaps in tumors. For example, intermittent changes in blood flow and sheer stress in the tumor vasculature may induce remodeling and IA on the time scale of minutes. Although IA does occur in the vasculature of growing tumors, it has been largely unexplored as a mechanism for creating new tumor blood vessels or as a process that could be inhibited to thwart tumor growth.

Despite initial enthusiasm that tumors can be killed by simply blocking angiogenesis, an idea pioneered and championed by the late Dr. Judah Folkman, the field of antiangiogenesis research in cancer has been met with some significant disappointments. For example, tumor vessels have proven to be more complex and labile than expected and it was not predicted that tumor might be cytogenetically abnormal or derived from multiple sources. Furthermore, there have been unexpected consequences of VEGF inhibition including an up-regulation of compensatory angiogenic pathways and increased metastasis in some mouse tumor models. Accordingly the current invention teaches methods of inducing immune responses towards tumor endothelial cells.

### SUMMARY

Preferred embodiments are directed to methods of inducing an immune response to tumor associated blood vessels comprising the steps of: a) obtaining a pluripotent stem cell; b) optionally modifying said pluripotent stem cell so as to induce expression of an immunogenic molecule; c) optionally modifying said pluripotent stem cell so as to induce loss of expression of an immunosuppressive molecule; d) optionally modifying said pluripotent stem cell so as to induce loss of expression of an immune suppressive signaling molecule; e) inducing differentiation of said pluripotent stem cell into endothelial cells under conditions which replicate the tumor microenvironment; f) obtaining said endothelial cells differentiated under conditions that replicate said tumor microenvironment and substantially isolating said cells in order to obtain a relatively homogeneous population of cells which resemble tumor endothelium associated cells; g) optionally mitotically inactivating said cells; and h) administering said cells in a manner to stimulate an immune response.

Preferred embodiments include methods wherein said immune response is an antibody mediated immune response.

Preferred embodiments include methods wherein said immune response is a cell mediated immune response.

Preferred embodiments include methods wherein said immune response is a natural killer cell mediated immune response.

Preferred embodiments include methods wherein said immune response is an NKT cell mediated immune response.

Preferred embodiments include methods wherein said immune response is a macrophage mediated immune response.

Preferred embodiments include methods wherein said tumor associated blood vessels are angiogenic.

Preferred embodiments include methods wherein said tumor associated blood vessels are vasculogenic.

Preferred embodiments include methods wherein said tumor associated blood vessels are comprised of proliferating cells.

Preferred embodiments include methods wherein said tumor associated blood vessels are comprised of proliferating endothelial cells.

Preferred embodiments include methods wherein said tumor associated blood vessels possess expression of TEM-1.

Preferred embodiments include methods wherein said tumor associated blood vessels possess expression of VEGF-receptor.

Preferred embodiments include methods wherein said tumor associated blood vessels possess expression of nestin.

Preferred embodiments include methods wherein said tumor associated blood vessels possess expression of TREM-1.

Preferred embodiments include methods wherein said tumor associated blood vessels possess expression of CD31.

Preferred embodiments include methods wherein said tumor associated blood vessels possess expression of vWF.

Preferred embodiments include methods wherein said tumor associated blood vessels possess expression of CD133.

Preferred embodiments include methods wherein said tumor associated blood vessels possess expression of CD34.

Preferred embodiments include methods wherein said tumor associated blood vessels possess expression of CD133.

Preferred embodiments include methods wherein said tumor associated blood vessels possess expression of Factor VIII.

Preferred embodiments include methods wherein said tumor associated blood vessels possess expression of c-met.

Preferred embodiments include methods wherein said tumor associated blood vessels possess expression of PDGF-BB receptor.

Preferred embodiments include methods wherein said tumor associated blood vessels possess expression of EGF-receptor.

Preferred embodiments include methods wherein said tumor associated blood vessels possess expression of FGF-1 receptor.

Preferred embodiments include methods wherein said tumor associated blood vessels possess expression of FGF-2 receptor.

Preferred embodiments include methods wherein said tumor associated blood vessels possess expression of klotho receptor.

Preferred embodiments include methods wherein said tumor associated blood vessels possess expression of netrin.

Preferred embodiments include methods wherein said tumor associated blood vessels possess expression of thrombopoietin receptor.

Preferred embodiments include methods wherein said tumor associated blood vessels possess expression of interleukin-3 receptor.

Preferred embodiments include methods wherein said tumor associated blood vessels possess expression of nestin.

Preferred embodiments include methods wherein said pluripotent stem cell is capable of forming a teratoma when placed in an immunodeficient mouse.

Preferred embodiments include methods wherein said pluripotent stem cell is capable of forming ectoderm, mesoderm and endoderm tissue.

Preferred embodiments include methods wherein said pluripotent stem cell is generated as an inducible pluripotent stem cell.

Preferred embodiments include methods wherein said pluripotent stem cell expresses OCT4.

Preferred embodiments include methods wherein said pluripotent stem cell expresses Sox-2.

Preferred embodiments include methods wherein said pluripotent stem cell expresses NANOG.

Preferred embodiments include methods wherein said pluripotent stem cell expresses SSEA4.

Preferred embodiments include methods wherein said pluripotent stem cell expresses c-myc.

Preferred embodiments include methods wherein said pluripotent stem cell expresses DNMT3B.

Preferred embodiments include methods wherein said pluripotent stem cell expresses KLF4.

Preferred embodiments include methods wherein said pluripotent stem cell expresses Lin28.

Preferred embodiments include methods wherein said pluripotent stem cell expresses PRDM14.

Preferred embodiments include methods wherein said pluripotent stem cell expresses SALL4.

Preferred embodiments include methods wherein said pluripotent stem cell expresses SSEA1.

Preferred embodiments include methods wherein said pluripotent stem cell expresses SSEA3.

Preferred embodiments include methods wherein said pluripotent stem cell expresses TRA-1-60.

Preferred embodiments include methods wherein said pluripotent stem cell expresses TRA-1-81.

Preferred embodiments include methods wherein said pluripotent stem cell is derived by somatic cell nuclear transfer.

Preferred embodiments include methods wherein said pluripotent stem cell is derived by parthenogenesis.

Preferred embodiments include methods wherein said pluripotent stem cell is derived by exposure to acid.

Preferred embodiments include methods wherein said pluripotent stem cell is transfected with an immune stimulatory cytokine whose expression is either constitutive or inducible.

Preferred embodiments include methods wherein said immune stimulatory cytokine is RANTES.

Preferred embodiments include methods wherein said immune stimulatory cytokine is MIP-1 alpha.

Preferred embodiments include methods wherein said immune stimulatory cytokine is MIP-1 beta.

Preferred embodiments include methods wherein said immune stimulatory cytokine is interleukin-1.

Preferred embodiments include methods wherein said immune stimulatory cytokine is interleukin-2.

Preferred embodiments include methods wherein said immune stimulatory cytokine is interleukin-6.

Preferred embodiments include methods wherein said immune stimulatory cytokine is interleukin-7.

Preferred embodiments include methods wherein said immune stimulatory cytokine is interleukin-8.

Preferred embodiments include methods wherein said immune stimulatory cytokine is interleukin-9.

Preferred embodiments include methods wherein said immune stimulatory cytokine is interleukin-12.

Preferred embodiments include methods wherein said immune stimulatory cytokine is interleukin-15.

Preferred embodiments include methods wherein said immune stimulatory cytokine is interleukin-17.

Preferred embodiments include methods wherein said immune stimulatory cytokine is interleukin-18.

Preferred embodiments include methods wherein said immune stimulatory cytokine is interleukin-20.

Preferred embodiments include methods wherein said immune stimulatory cytokine is interleukin-22.

Preferred embodiments include methods wherein said immune stimulatory cytokine is TNF-alpha.

Preferred embodiments include methods wherein said immune stimulatory cytokine is TNF-beta.

Preferred embodiments include methods wherein said immune stimulatory cytokine is interferon alpha.

Preferred embodiments include methods wherein said immune stimulatory cytokine is interferon beta.

Preferred embodiments include methods wherein said immune stimulatory cytokine is interferon gamma.

Preferred embodiments include methods wherein said immune stimulatory cytokine is interferon epsilon.

Preferred embodiments include methods wherein said immune stimulatory cytokine is interferon tau.

Preferred embodiments include methods wherein said immune stimulatory cytokine is interferon omega.

Preferred embodiments include methods wherein said immune stimulatory cytokine is CD80.

Preferred embodiments include methods wherein said immune stimulatory cytokine is CD86.

Preferred embodiments include methods wherein said immune stimulatory cytokine is CD40.

Preferred embodiments include methods wherein said immune stimulatory cytokine is CD40 ligand.

Preferred embodiments include methods wherein said immune stimulatory cytokine is CD2.

Preferred embodiments include methods wherein said immune stimulatory cytokine is CD5.

Preferred embodiments include methods wherein said immune stimulatory cytokine is CD28.

Preferred embodiments include methods wherein said immune stimulatory cytokine is Fas ligand.

Preferred embodiments include methods wherein said immune stimulatory cytokine is G-CSF.

Preferred embodiments include methods wherein said immune stimulatory cytokine is GM-CSF.

Preferred embodiments include methods wherein said immune stimulatory cytokine is M-CSF.

Preferred embodiments include methods wherein said immune stimulatory cytokine is lymphotactin.

Preferred embodiments include methods wherein said immune stimulatory cytokine is SDF-1.

Preferred embodiments include methods wherein said pluripotent stem cell is transfected with alpha1,3-galactosyltransferase.

Preferred embodiments include methods wherein said pluripotent stem cell is gene edited to remove expression of IL-10 receptor.

Preferred embodiments include methods wherein said pluripotent stem cell is gene edited to remove expression of IL-4 receptor.

Preferred embodiments include methods wherein said pluripotent stem cell is gene edited to remove expression of IL-3 receptor.

Preferred embodiments include methods wherein said pluripotent stem cell is gene edited to remove expression of NGF receptor.

Preferred embodiments include methods wherein said pluripotent stem cell is gene edited to remove expression of TNF receptor p55.

Preferred embodiments include methods wherein said pluripotent stem cell is gene edited to remove expression of TNF receptor p75.

Preferred embodiments include methods wherein said endothelial cells are generated by culture for pluripotent stem cells in a media inductive of CD133 generation.

Preferred embodiments include methods wherein said pluripotent stem cells are cultured in PDGF-BB to generate endothelial progenitor cells.

Preferred embodiments include methods wherein said pluripotent stem cells are cultured in PDGF-BB to generate endothelial progenitor cells.

Preferred embodiments include methods wherein said immunogenicity is augmented by fusion of human pluripotent cell with a cell from an animal that is not a human or a primate.

Preferred embodiments include methods wherein said endothelial cells are generated from said pluripotent cell by: a) culturing or maintaining a plurality of substantially undifferentiated pluripotent cells in a defined media comprising at least one growth factor; b) culturing the pluripotent cells in a defined media comprising an amount of BMP4 and VEGF sufficient to expand or promote differentiation in a plurality of the cells; and c) culturing the cells of (b) in a defined media comprising an amount of either (1) IL-3 and Flt3 ligand, or (2) VEGF, FGF-2 or an FGF-2 mimic, and IGF sufficient to further expand or promote differentiation in a plurality of the cells; wherein a plurality of the pluripotent cells are differentiated into hematopoietic precursor cells or endothelial cells.

Preferred embodiments include methods wherein the defined media of step (b) further comprises FGF-2 or an FGF-2 mimic.

Preferred embodiments include methods methods wherein the defined media of step (c) comprises IL-3, Flt3 ligand, and GMCSF.

Preferred embodiments include methods wherein the defined media of step (c) comprises IL-3, Flt3 ligand, and at least one of IL-6, SCF, or TPO.

Preferred embodiments include methods wherein the defined media of step (c) further comprises IL-6, SCF, and TPO.

### BRIEF DESCRIPTION OF THE DRAWINGS

FIG. 1 is a bar graph showing iPSCs expressing α-Gal epitope were successful in cellular death.

### DESCRIPTION OF THE INVENTION

The invention provides means of inducing immune responses towards tumor endothelial cells through immunization with pluripotent stem cell derived endothelial cells generated in a manner to replicate immunogenic characteristics of tumor endothelial cells. In one embodiment inducible pluripotent stem cells are modified to increase immunogenicity and differentiated under conditions of hypoxia, acidosis, tryptophan depletion and other conditions known to be associated with the tumor microenvironment [1,2]. Said iPSC generated tumor endothelial cells (iPSC-TEC) are made immunogenic by inducing expression of the α-Gal epitope (Galα1,3Galα1,4G1cNAc-R).

A media is said to be “essentially free” of a growth factor if the growth factor is absent from the media or if the growth factor is present in the media in an amount which is insufficient to promote any substantial expansion and/or differentiation of cells in the media, or is present at a concentration below a detectable limit. It will be recognized that a media which is essentially free of a growth factor may nonetheless contain trace amounts of the growth factor in the media.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

An aspect of the present invention relates to a method of differentiating pluripotent cells into hematopoietic precursor cells or endothelial cells comprising the sequential steps of: (a) culturing or maintaining a plurality of substantially undifferentiated pluripotent cells in a first defined media comprising at least one growth factor, (b) incubating the cells in a second defined media which is essentially free of BMP4, VEGF, IL-3, Flt3 ligand, and GMCSF, (c) culturing the cells in a third defined media comprising an amount of BMP4 and VEGF sufficient to expand or promote differentiation in a plurality of the cells, and (d) culturing the cells in a fourth defined media comprising an amount of either (1) IL-3 and Flt3 ligand, or (2) VEGF, FGF-2 or an FGF-2 mimic, and IGF sufficient to expand or promote differentiation in a plurality of the cells; wherein a plurality of the pluripotent cells are differentiated into hematopoietic precursor cells or endothelial cells. In certain embodiments, combination (1) above may be used to promote differentiation into hematopoietic precursor cells. Combination (2) above may be used to promote differentiation into an endothelial cell or an endothelial progenitor cell. The second defined media may be free or essentially free of FGF-2, IL6, SCF and/or TPO. The third defined media may also include FGF-2 (e.g., from about 5-50 ng/ml or from about 10-25 ng/ml) or an FGF-2 mimic. As shown in the below examples, inclusion of FGF-2 in the third media can increase the efficiency of differentiation of pluripotent cells into hematopoietic precursor cells. In certain embodiments, the fourth defined media further comprises GMCSF, or at least one of IL-6, SCF, or TPO. In certain embodiments, the fourth defined media includes an amount of either: (1) IL-3, Flt3 ligand, and GMCSF, or (2) IL-3, Flt3 ligand, SCF, IL-6, and TPO sufficient to promote differentiation of the cells. The third defined media and/or the fourth defined media may further comprise BIT9500 or Serum Replacement 3. The method may comprise culturing cells in a defined media which includes BIT9500 or Serum Replacement 3. At least some of the cells may be at least partially separated or are substantially individualized prior to step (b). The cells may be substantially individualized using an enzyme, such as a trypsin. The cells may be contacted with a ROCK inhibitor and a trypsin inhibitor (e.g., a soybean trypsin inhibitor) subsequent to said individualization. The ROCK inhibitor may be selected from the list consisting of HA-100, H-1152, and Y-27632. A plurality of the pluripotent cells may form embryoid bodies (EBs). From about 200 to about 1000 cells per aggregate may be used to generate at least one of said EB s. The method may comprise culturing the cells at an atmospheric pressure of less than 20% oxygen or at an atmospheric pressure of about 5% oxygen. As shown in the below examples, differentiating cells under hypoxic conditions, such as at about 5% atmospheric O.sub.2, can increase the differentiation of the cells, e.g., into hematopoietic and/or endothelial precursor cells.

In certain embodiments, said cells may be partially or substantially reaggregated at least once. The cells may be reaggregated after culture in the third defined media and prior to or during culture in the fourth defined media. The reaggregation may comprise exposing said cells to trypsin or TRYPLE. Said cells may be exposed to a ROCK inhibitor subsequent to the reaggregation, or said cells may be cultured in a media essentially free of a ROCK inhibitor subsequent to the reaggregation. The method may further comprise culturing the cells at an atmospheric pressure of less than about 20% oxygen, wherein from about 200 to about 1000 cells per aggregate are used to generate a plurality of embryoid bodies (EBs). The first defined media may comprise TeSR, mTeSR, or mTeSR1. Step (a) may comprise culturing the cells on a matrix-coated surface. The matrix may comprise laminin, vitronectin, gelatin, polylysine, thrombospondin or Matrigel.TM.. The second defined media may comprise TeSR-GF or X-vivol5 media. The second defined media may further comprise about 0.1 ng/ml TGF-.beta. and about 20 ng/ml FGF-2. Step (b) may comprise incubating the cells for a period of from about 12 hours to about 3 days. Step (c) may comprise culturing or differentiating the cells for a period of from about 4 to about 8 days. Step (d) may comprise culturing the cells for a period of at least about 4, or from about 4 to about 8 days. A plurality of the pluripotent cells may be differentiated into multipotent heamatopoietic, or myeloid progenitor cells. In certain embodiments, the myeloid progenitor cells co-express CD31, CD43, and CD45. The myeloid progenitor cells may be common myeloid progenitors. The third defined media comprises about 10-50 ng/ml BMP4 and about 10-50 ng/ml VEGF. In certain embodiments, the third defined media further comprises 10-50 ng/ml FGF-2. The third defined media comprises about 25 ng/ml BMP4 and about 25 ng/ml VEGF. The fourth defined media may comprise about 5-25 ng/ml IL-3 and about 10-50 ng/ml Flt3 ligand. The fourth defined media may further comprise about 5-25 ng/ml GMCSF, or about 10-100 ng/ml or about 10-50 ng/ml TPO, about 10-100 ng/ml SCF, about 5-25 ng/ml IL-6, and about 5-25 ng/ml IL-3. The fourth defined media may comprise about 10 ng/ml IL-3, about 25 ng/ml Flt3 ligand, and about 10 ng/ml GMCSF. A plurality of the hematopoietic precursor cells may express at least two cell markers selected from the list comprising CD43, CD34, CD31 and CD45. A plurality of the hematopoietic precursor cells may express CD34, CD43, CD45 and CD31. In certain embodiments, the hematopoietic precursor cells are multipotent hematopoietic precursor cells that co-express CD34, CD43, CD45 and CD31. In certain embodiments, a fifth defined media may be used to further promote differentiation of the cells into a particular cell type; for example, various media may be used to promote differentiation of the hematopoietic precursor cells into a more differentiated cell type such as, for example, an erythroblast, a NK cell, or a T cell. The method may further comprise culturing a plurality of said cells in a fifth defined media comprising one or more growth factor selected from the list consisting of IL-3, IL-6, SCF, EPO, and TPO, in an amount sufficient to promote differentiation of a plurality of the cells into erythroblasts. A plurality of the cells are cultured in a fifth defined media comprising one or more growth factor selected from the list consisting of IL-7, SCF, and IL-2, in an amount sufficient to promote differentiation of the cells into NK cells. The method may further comprise culturing a plurality of said cells in a fifth defined media comprising Notch ligand and one or more growth factor selected from the list consisting of IL-7, SCF, and IL-2 in an amount sufficient to promote differentiation of the cells into T cells. The Notch ligand may be the Fc chimeric Notch DLL-1 ligand or Notch ligand produced by a stromal cell line which over-expresses the Notch ligand. In certain embodiments, a thymic peptide such thymosin alpha, thymopenin, or thymosin B4 may be used to further promote differentiation of the cells into T cells (e.g., as described in Peng et al., 2008). In certain embodiments, the plurality of said cells comprise hematopoietic precursor cells. The third defined media may comprise one or more growth factor selected from the list consisting of SCF, IL-6, G-CSF, EPO, TPO, FGF2, IL-7, IL-11, IL-9, IL-13, IL-2, or M-CSF in an amount sufficient to promote expansion or further differentiation of the cells. The fourth defined media may comprise one or more growth factor selected from the list consisting of SCF, IL-6, G-CSF, EPO, TPO, FGF2, BMP4, VEGF, IL-7, IL-11, IL-9, IL-13, IL-2, or M-CSF in an amount sufficient to promote expansion or further differentiation of the cells. In certain embodiments, the method may comprise incubating the cells in a fifth defined media which includes one or more growth factor selected from the list consisting of SCF, IL-6, G-CSF, EPO, TPO, FGF2, IL-7, IL-11, IL-9, IL-13, IL-2, or M-CSF in an amount sufficient to promote expansion or further differentiation of the cells. Said pluripotent cells are preferably mammalian pluripotent cells. In certain embodiments the pluripotent cells are human pluripotent cells, such as human embryonic stem cells (hESC) or induced pluripotent cells (iPSC). The hESC comprise cells may be selected from the list consisting of H1, H9, hES2, hES3, hES4, hES5, hES6, BG01, BG02, BG03, HSF1, HSF6, H1, H7, H9, H13B, and H14. Said iPSC may be selected from the list consisting of iPS6.1, iPS 6.6, iPS, iPS 5.6, iPS iPS 5.12, iPS 5.2.15, iPS iPS 5.2.24, iPS 5.2.20, iPS 6.2.1, iPS-B 1-SONL, iPS-B1-SOCK, iPS-TIPS 1EE, iPS-TiPS IB, iPS-KIPS-5, and iPS 5/3-4.3. Another aspect of the present invention related to hematopoietic precursor cell differentiated according to the methods described herein or derived from a separate hematopoietic precursor cell differentiated according to the methods described herein. The hematopoietic precursor cell may express two, three or all of CD34, CD43, CD45, and CD31. Yet another aspect of the present invention relates to a myeloid cell, a myeloid progenitor, or a common myeloid progenitor derived from a hematopoietic precursor cell differentiated according to the methods described herein. The myeloid cell may be selected from the list consisting of monocyte, macrophage, neutrophil, basophil, eosinophil, erythrocyte, megakaryocyte/platelet, and dendritic cell. In certain embodiments, the myeloid cell is an erythrocyte. The myeloid cell, myeloid progenitor, or the common myeloid progenitor may be comprised in a pharmaceutical preparation.

In one embodiment of the invention, administration of pluripotent stem cell derived tumor endothelial cells (iPSC-TEC) is performed in the presence of other immune activators. It is known from studies of immune modulators that recruitment of multiple arms of the immune system associates with increased efficacy. Accordingly, in one embodiment of the invention, numerous anti-cancer immune effector cells are stimulated together with administration of the iPSC-TEC immunotherapy. It is known that natural killer cells play an important role in immune destruction of cancer [3-9]. In one embodiment iPSC-TEC are administered together with activators of NK cells. Known NK activators useful for the practice of the invention include interferon alpha [10-12], interferon gamma [13], OK-432 [14-24]. The importance of NK activation can be seen in numerous publications which guide one of skill in the art on the practice of the invention. For example, a clinical trial demonstrated that patients who possess elevated levels of natural killer cell inhibitory proteins (soluble NKG2D ligands) demonstrated lower responses to checkpoint inhibitors [25]. Indeed, this should not be surprising since studies show that NK cell infiltration of tumors induces upregulation of antigen presentation in an interferon gamma associated manner, which renders tumor cells sensitive to T cell killing [26]. In one embodiment of the invention iPSC-TEC are utilized together with NK cell activators and checkpoint inhibitors. In other embodiments iPSC-TEC are combined with standard immunotherapies. In one embodiment said iPSC-TEC potently induce destruction of tumor blood vessels, wherein said damaged tumor blood vessels increase ability of immune cells to enter the tumor. Another example of the potency of combining immunotherapies is the example of Herceptin, in which approximately 1 out of 4 patients with the HER2neu antigen respond to treating. Interestingly it was found that lack of responsiveness correlates with inhibited NK cell activity [27-29]. Indeed, animal experiments demonstrate augmentation of Herceptin activity by stimulators of NK cells such as Poly (IC) and IL-12 [30, 31].

In one embodiment of the invention, iPSC-TEC are utilized together with macrophage activating means, and/or with activation M1 macrophage administration. Macrophages are key components of the innate immune system which play a principal role in the regulation of inflammation as well as physiological processes such as tissue remodeling [32,33]. The diverse role of macrophages can be seen in conditions ranging from wound healing [34-37], to myocardial infarction [38-44], to renal failure [45-48] and liver failure [49].

Differentiated macrophages and their precursors are versatile cells that can adapt to micro environmental signals by altering their phenotype and function [50]. Although they have been studied for many years, it has only recently been shown that these cells comprise distinct sub-populations, known as classical M1 and alternative M2 [51]. Mirroring the nomenclature of Th1 cells, M1 macrophages are described as the pro-inflammatory sub-type of macrophages induced by IFN-.gamma. and LPS. They produce effector molecules (e.g., reactive oxygen species) and pro-inflammatory cytokines (e.g., IL-12, TNF-.alpha. and IL-6) and they trigger Th1 polarized responses [52]. Macrophages can play a tumor inhibitory, as well as a tumor stimulatory role. Initial studies supported the role of macrophages in mediating antibody dependent cellular cytotoxicity in tumors [53-60], and thus being associated with potentiation of antitumor immune responses. Macrophages also possess the ability to directly recognize tumors by virtue of tumor expressed “eat-me” signals, which include the stress associated protein calreticulin [61,62], which binds to the low-density lipoprotein receptor-related protein (LRP) on macrophages to induce phagocytosis [63]. Tumors protect themselves by expression of CD47, which binds to macrophage SIRP-1 and transduces an inhibitory signal [64]. Blockade of CD47 using antibodies results in remission of cancers mediated by macrophage activation [65-69]. Thus on the one hand, macrophages play an important role in induction of antitumor immunity. This can also be exemplified by some studies, involving administration of GM-CSF in order to augment macrophage numbers and activity in cancer patients [70-73].

Unfortunately, there is also evidence that macrophages support tumor growth. Accordingly, for the practice of the invention, care must be taken to inhibit the tumor promoting activities of macrophages. Studies in the osteoporotic mice strain, which lacks mature macrophages, demonstrate that tumors actually grow slower in animals deficient in macrophages [74]. Several other animal models have elegantly demonstrated that macrophages contribute to tumor growth, in part through stimulating on the angiogenic switch [75-77]. Numerous tumor biopsy studies have shown that there is a negative correlation between macrophage infiltration into tumors and patient survival [78-82]. The duality of macrophages in growth of tumors may be seen in studies of “inverse hormesis” in which low concentrations of antibodies targeting the tumor specific marker sialic acid N-glycolyl-neuraminic acid (Neu5Gc) actually leads to enhanced tumor growth in a macrophage dependent manner [83].

The importance of macrophages in clinical implementation of cancer therapeutics can be seen from results of a double blind clinical trials in metastatic colorectal cancer patients where cetuximab (anti-epidermal growth factor receptor (EGFR) monoclonal antibody (mAb)) was added to a protocol comprising of bevacizumab and chemotherapy. The addition of cetuximab actually resulted in decreased survival. In a study examining whether monocyte conversion to M2 angiogenic macrophages was responsible, investigators observed that CD163-positive M2 macrophages where found in high concentrations within the tumors of patients with colorectal carcinomas. These M2 cells expressed abundant levels of Fc-gamma receptors (FcγR) and PD-L1. Additionally, consistent with the M2 phenotype the cells generated large amounts of the immunosuppressive molecule IL-10 and the angiogenic mediator VEGF. When M2 cells were cultured with EGFR-positive tumor cells loaded with low concentrations of cetuximab, further augmentation of IL-10 and VEGF production was observed. These data suggest that under certain contexts, tumors manipulate macrophages to take on the M2 phenotype, and this subsequently leads to enhanced tumor progressing factors when tumor cells are bound by antibodies [84].

Manipulation of macrophages to inhibit M2 and shift to M1 phenotype may be performed using a variety of means. One theme that seems unifying is the ability of toll like receptor (TLR) agonists to influence this. In addition to cytokine differences, macrophages capable of killing tumor cells are usually known to express low levels of the inhibitory Fc gamma receptor IIb, whereas tumor promoting macrophages have high levels of this receptor [85]. Furthermore, tumor associated cytokines such as IL-4 and IL-10 are known to induce upregulation of the Fc gamma receptor IIB [86-89].

In one embodiment of the invention, iPSC-TEC are utilized together with agents and means that activate T cell responses. In one embodiment said T cell responses are utilized to kill tumor endothelial associated molecules. In other embodiments iPSC-TEC are utilized to enhance therapeutic ability of tumor infiltrating T cells. It is known that T cells are immune effectors against tumors, possessing ability to directly kill via CD8 cytotoxic cells [90-92], or indirectly killing tumors by activation of macrophages through interferon gamma production [93-95]. Additionally, T cells have been shown to convert pro-tumor M2 macrophages to M1 [96]. The importance of T cells in cancer is illustrated by positive correlation between tumor infiltrating lymphocytes and patient survival [97-101]. In addition, positive correlations between responses to various immunotherapies has been made with tumor infiltrating lymphocyte density [102, 103]. Increased T cell activity is associated with reduction in T regulatory (Treg) cells. Studies show that agents that cause suppression of Treg cells correlates with improved tumor control. Agents that inhibit Treg cells, which can be utilized together with iPSC-TEC include arsenic trioxide [104], cyclophosphamide [105-107], triptolide [106], gemcitabine [108], and artemether [109].

In one embodiment of the invention, Angiogenesis, the outgrowth of new blood vessels from pre-existing capillaries and post-capillary venules, occurs during embryonic development, in the uterus during the menstrual cycle, in the process of wound healing, and in pathological conditions [110]. In healthy adults, endothelial cells can maintain a quiescent state for years, whereas they proliferate and migrate to form new vessels in response to inflammatory conditions and during tumor growth. Studies have estimated as much as 30-40 fold more rapid proliferation of endothelial cells in tumors vs. normal vasculature [111-113]. Based on estimates that tumors fail to grow beyond 1-2 mm in the absence of new capillary growth, Dr. Judah Folkman put forth the central hypothesis that tumors release diffusible factors that stimulate endothelial cell proliferation in host capillary blood vessels [114]. Indeed, it has been estimated that eradication of one endothelial cell is capable of neutralizing of up to 100-300 tumor cells [115]. Since the immune system is in direct contact with the tumor vasculature, vaccination against endothelium is theoretically very promising for breaching the barriers created by the tumor microenvironment.

The goal in vaccination strategies is to raise immunity against antigens present in tumor endothelium while avoiding antigens that cross-react with healthy vasculature, thereby preventing deleterious autoimmune reactions. Since the landmark publication by Dr. Folkman, a catalog of molecular players involved in the process of tumor angiogenesis have been identified and characterized. Clinical outcomes of traditional anti-angiogenic therapies such as monoclonal antibodies have improved patient survival rates only modestly [116]. Vaccination against endothelial cells is poised to overcome the existing problems of drug resistance and adverse side effects associated with other approaches. This report reviews vaccination strategies against the tumor endothelium that have been tested to date, including DNA, protein and peptide vaccines using tumor-endothelium-associated antigens, as well as polyvalent vaccines comprising whole endothelial cells. Very encouraging data point toward the efficacy of vaccination in raising humoral and cell-mediated immunity against angiogenesis-associated antigens in cancer. Numerous approaches have been developed in attempts to selectively block tumor angiogenesis or induce collapse of tumor-associated blood vessels. While initial attempts such as development of endogenous inhibitors such as angiostatin and endostatin have failed, immunological means such as passive antibodies to VEGF (Avastin) have had success in terms of regulatory clearance and marketing approval. Drawbacks of Avastin include cardiotoxicity, development of resistance, has well as relatively poor survival advantage. Conceptually a more appealing method of inducing angiogenesis blockade would involve active immunization against several tumor endothelial associated antigens in the form of a polyvalent vaccine.

One major question that arises during attempts to induce active immunity to tumor associated endothelial is the “horror autotoxicus” potential of stimulating immunity towards non-malignant endothelium. We recently reviewed numerous works in which immunization to proliferating endothelial cells, whether syngeneic, allogeneic or xenogeneic results in selectivity of killing of tumors without damage to non-malignant tissues [117]. This is a fundamental point because numerous antigens found on tumor endothelial cells are also found on non-malignant cells, for example VEGFR is known to be associated with hematopoietic stem cell self-renewal. Despite this, as reviewed, immunization with VEGFR protein or plasmid does not result in ablation of hematopoietic stem cells, as would be expected. Accordingly multiple mechanisms must be at operation that discriminate tumor endothelial from cells expressing similar markers but are not under immunological attack and destruction as a result of the immunization. This is supported by clinical data in which immunization with HUVEC cells multiple times did not result in hematopoietic or other toxicities. While numerous attempts have been made at immunizing tumor bearing mice to endothelial antigens, the mechanistic data behind development of immunity has not been elucidated. Wei et al, for example, demonstrated involvement of antibodies targeting alpha V beta III integrin in suppression of tumor angiogenesis following immunization of mice with xenogeneic HUVEC cells [118]. Other studies have implicated T cell responses [119-121]. Indeed, in some situations not only collaboration between T cells and B cells is required for successful antitumor immunity, but also epitope spreading is observed between initial immunity towards tumor endothelium, which is subsequently followed by immunity towards tumor antigens themselves. Thus there is a high degree of variability of biological mechanisms between different active immunotherapies which target the tumor vasculature.

In some embodiments of the invention, human placental endothelial cell derived cellular vaccine product is used as a substitute for iPSC-TEC for vaccination. A similar vaccine has demonstrated human safety in an initial pilot clinical trial [122], as well as being shown to effectively reduce tumor growth in lung cancer, melanoma, and breast cancer [123]. In contrast to other endothelium based vaccines. Generating cancer endothelial targeting immunotherapies using placenta as a source of tissue has the unique properties of: a) Large donor supply. Since the vaccine is generated from placental endothelial cells, there exists a virtually unlimited supply of placentas, and additionally, each placenta is capable of generating a large number of doses; b) The vaccine is optimized for immunogenicity by pre-treatment with stimulators of HLA and CD80/86; and c) Placental endothelial is biologically naïve, thus allowing for a higher degree of plasticity. The enhanced plasticity allows for higher levels of surface marker manipulation subsequent to treatment with cytokines.

For generation of immunogenic cells, iPSC cells may be differentiated into cells that resemble endothelial cells by culture in conditions such as certain growth factors are particularly important for the differentiation of pluripotent cells which have been maintained under defined conditions. In certain embodiments, pluripotent cells may be sequentially exposed to several defined media to promote differentiation into hematopoietic precursor cells. After culture and maintenance of the pluripotent cells in an essentially undifferentiated state in a first defined media (e.g., in a TeSR media), the cells may be exposed to a second defined media containing no or essentially no BMP4, VEGF, IL-3, Flt3 ligand, or GMCSF. The cells may then be exposed to a third defined media comprising BMP4, VEGF, IL-3, Flt3 ligand, and GMCSF to promote hematopoietic differentiation; alternately, the cells may be exposed to a third defined media comprising BMP4 and VEGF, and optionally FGF-2; followed by exposure to a fourth media comprising IL-3, Flt3 ligand, and GMCSF. The inventor has discovered that sequential exposure to a third defined media comprising BMP4 and VEGF, followed by exposure to a fourth media comprising IL-3, Flt3 ligand, and GMCSF can surprisingly result in substantial increases in the generation of hematopoietic precursor cells. As shown in the below examples, inclusion of FGF-2 in the third defined media resulted in a surprising increase in the differentiation of pluripotent cells into hematopoietic precursor cells. It has also been discovered that hypoxic conditions (e.g., exposure to an atmospheric pressure 5% O.sub.2), at least partial reaggregation of cells (e.g., using trypsin or TrypLE.TM.), and/or formation of aggregates using defined ranges of cells in the formation of embryoid bodies (e.g., from about 200-1000 cells per aggregate) can also be used to further promote differentiation into hematopoietic precursor cells. Endothelial cells may be generated, for example, using the following protocol for implantation into an animal or human subject. Human cell-derived CD31+ cells may be cultured in either EGM.TM.-2 medium (Lonza, Switzerland) or differentiation medium with 50 ng/mL rhVEGF and 5 ng/mL rhFGF-2 for 7 to 10 days. For further expansion and differentiation of endothelial cells, isolated CD31+ cells may be cultured in endothelial differentiation medium (Lonza catlalog #CC3202) containing VEGF, FGF, EGF, IGF, ascorbic acid and FBS or differentiation medium containing 50 ng/ml, vasular endothelial growth factor (VEGF), and FGF-2 (e.g., 50 ng/ml zebrafish FGF-2). Cells may be cultured for 2 to 3 weeks.] The following protocol may be used to induce endothelial differentiation. For expansion and differentiation of endothelial cells, isolated CD34+ cells may be seeded on gelatin-coated wells (1.5 to 2.times.10.sup.4 cells/cm.sup.2) in EGM-2MV medium (Cambrex). Collagen I-coated wells (BD labware) may also be used, although gelatin is typically less expensive than collagen I-coated wells. CD34+ cells may be cultured in hESC differentiation medium containing the endothelial growth factors, hVEGF.sub.165 (50 ng/mL) and FGF-2 (5 ng/mL). After about 7-10 days of incubation, the adherent cells may be harvested by trypsin-treatment and used for analyses. Endothelial cells may be evaluated by imaging a Matrigel plug. For example, the following protocol may be used to image cells: endothelial cells (ECs) derived from hESCs or iPSCs may be suspended in Matrigel (e.g., about 1 million cells in 1 ml) and injected subcutaneously into SCID Beige mice. FITC Dextran can then be injected intravenously at about day 14 before the removal of the matrigel plug. The plugs may be harvested and subjected to imaging using fluorescence imaging techniques, and the plug may be analyzed for the presence or absence of neovascularization.

### Example

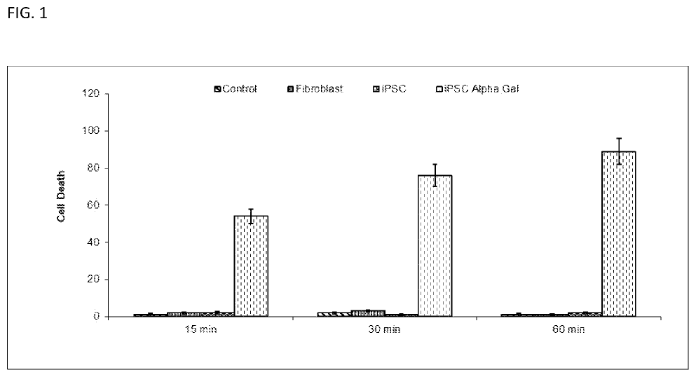

iPSC expressing α-Gal epitope (Galα1,3Galα1,4G1cNAc-R) where differentiated into tumor endothelial like cells by culture in VEGF (10 ng/ml), PDGF-BB (5 ng/ml), PGE-2 (100 pg/ml). Cells were incubated with human plasma and assessed for viability using MTT assay. Results are shown in FIG. 1.

### REFERENCES

1. Huang, M., et al., Macrophage inhibitory cytokine-1 induced by a high-fat diet promotes prostate cancer progression by stimulating tumor-promoting cytokine production from tumor stromal cells. Cancer Commun (Lond), 2021.
2. Bareche, Y., et al., High-dimensional analysis of the adenosine pathway in high-grade serous ovarian cancer. J Immunother Cancer, 2021. 9(3).
3. Martin-Fontecha, A., et al., Induced recruitment of NK cells to lymph nodes provides IFN-gamma for T(H)1 priming. Nat Immunol, 2004. 5(12): p. 1260-5.
4. Morandi, B., et al., NK cells of human secondary lymphoid tissues enhance T cell polarization via IFN-gamma secretion. Eur J Immunol, 2006. 36(9): p. 2394-400.
5. Ksienzyk, A., et al., IRF-1 expression is essential for natural killer cells to suppress metastasis. Cancer Res, 2011. 71(20): p. 6410-8.
6. Lopez-Soto, A., et al., Control of Metastasis by NK Cells. Cancer Cell, 2017. 32(2): p. 135-154.
7. Krasnova, Y., et al., Bench to bedside: NK cells and control of metastasis. Clin Immunol, 2017. 177: p. 50-59.
8. Putz, E. M., et al., NK cell heparanase controls tumor invasion and immune surveillance. J Clin Invest, 2017. 127(7): p. 2777-2788.
9. Morvan, M. G. and L. L. Lanier, NK cells and cancer: you can teach innate cells new tricks. Nat Rev Cancer, 2016. 16(1): p. 7-19.
10. Kozlowska, A., J. Mackiewicz, and A. Mackiewicz, Therapeutic gene modified cell based cancer vaccines. Gene, 2013. 525(2): p. 200-7.
11. Hervas-Stubbs, S., et al., Direct effects of type I interferons on cells of the immune system. Clin Cancer Res, 2011. 17(9): p. 2619-27.
12. Novick, D., et al., The neutralization of type I IFN biologic actions by anti-IFNAR-2 monoclonal antibodies is not entirely due to inhibition of Jak-Stat tyrosine phosphorylation. J Interferon Cytokine Res, 2000. 20(11): p. 971-82.
13. Gonsky, R., et al., Enhancer role of STATS in CD2 activation of IFN-gamma gene expression. J Immunol, 2004. 173(10): p. 6241-7.
14. Pan, K., et al., OK-432 synergizes with IFN-gamma to confer dendritic cells with enhanced antitumor immunity. Immunol Cell Biol, 2014. 92(3): p. 263-74.
15. Chen, I. J., et al., Vaccination with OK-432 followed by TC-1 tumor lysate leads to significant antitumor effects. Reprod Sci, 2011. 18(7): p. 687-94.
16. Takahashi, E., et al., Induction of CD16+CD56bright NK cells with antitumour cytotoxicity not only from CD16-CD56bright NK Cells but also from CD16-CD56dim NK cells. Scand J Immunol, 2007. 65(2): p. 126-38.
17. Okinaga, K., et al., Effect of immunotherapy and spleen preservation on immunological function in patients with gastric cancer. J Exp Clin Cancer Res, 2006. 25(3): p. 339-49.
18. Sato, M., et al., Generation of mature dendritic cells fully capable of T helper type 1 polarization using OK-432 combined with prostaglandin E(2). Cancer Sci, 2003. 94(12): p. 1091-8.
19. Okamoto, M., et al., Enhancement of anti-tumor immunity by lipoteichoic acid-related molecule isolated from OK-432, a streptococcal agent, in athymic nude mice bearing human salivary adenocarcinoma: role of natural killer cells. Anticancer Res, 2002. 22(6A): p. 3229-39.
20. Uehara, K., et al., Systemic administration of liposome-encapsulated OK-432 prolongs the survival of rats with hepatocellular carcinoma through the induction of IFN-gamma-producing hepatic lymphocytes. J Gastroenterol Hepatol, 2002. 17(1): p. 81-90.
21. Okamoto, M., et al., Induction of Th1-type cytokines by lipoteichoic acid-related preparation isolated from OK-432, a penicillin-killed streptococcal agent.

Immunopharmacology, 2000. 49(3): p. 363-76.
22. Yamagiwa, S., et al., Liposome-encapsulated OK-432 specifically and sustainedly induces hepatic natural killer cells and intermediate T cell receptor cells. J Gastroenterol Hepatol, 2000. 15(5): p. 542-9.
23. Yamamoto, K., et al., Effects of OK-432 on the proliferation and cytotoxicity of lymphokine-activated killer (LAK) cells. J Immunother, 1999. 22(1): p. 33-40.
24. Kurosawa, S., et al., The concurrent administration of OK432 augments the antitumor vaccination effect with tumor cells by sustaining locally infiltrating natural killer cells. Cancer Immunol Immunother, 1996. 43(1): p. 31-8.
25. Maccalli, C., et al., Soluble NKG2D ligands are biomarkers associated with the clinical outcome to immune checkpoint blockade therapy of metastatic melanoma patients. Oncoimmunology, 2017. 6(7): p. e1323618.
26. Goding, S. R., et al., Adoptive transfer of natural killer cells promotes the anti-tumor efficacy of T cells. Clin Immunol, 2017. 177: p. 76-86.
27. Muraro, E., et al., Improved Natural Killer cell activity and retained anti-tumor CD8(+) T cell responses contribute to the induction of a pathological complete response in HER2-positive breast cancer patients undergoing neoadjuvant chemotherapy. J Transl Med, 2015. 13: p. 204.
28. Lee, S. C., et al., Natural killer (NK): dendritic cell (DC) cross talk induced by therapeutic monoclonal antibody triggers tumor antigen-specific T cell immunity. Immunol Res, 2011. 50(2-3): p. 248-54.
29. Beano, A., et al., Correlation between NK function and response to trastuzumab in metastatic breast cancer patients. J Transl Med, 2008. 6: p. 25.
30. Jaime-Ramirez, A. C., et al., IL-12 enhances the antitumor actions of trastuzumab via NK cell IFN-gamma production. J Immunol, 2011. 186(6): p. 3401-9.
31. Charlebois, R., et al., Polyk C and CpG Synergize with Anti-ErbB2 mAb for Treatment of Breast Tumors Resistant to Immune Checkpoint Inhibitors. Cancer Res, 2017. 77(2): p. 312-319.
32. van Furth, R. and Z. A. Cohn, The origin and kinetics of mononuclear phagocytes. J Exp Med, 1968. 128(3): p. 415-35.
33. Wynn, T. A., A. Chawla, and J. W. Pollard, Macrophage biology in development, homeostasis and disease. Nature, 2013. 496(7446): p. 445-55.
34. Smith, T. D., et al., Harnessing macrophage plasticity for tissue regeneration. Adv Drug

Deliv Rev, 2017.
35. Vannella, K. M. and T. A. Wynn, Mechanisms of Organ Injury and Repair by Macrophages. Annu Rev Physiol, 2017. 79: p. 593-617.
36. Boddupalli, A., L. Zhu, and K. M. Bratlie, Methods for Implant Acceptance and Wound Healing: Material Selection and Implant Location Modulate Macrophage and Fibroblast Phenotypes. Adv Healthc Mater, 2016. 5(20): p. 2575-2594.
37. Snyder, R. J., et al., Macrophages: A review of their role in wound healing and their therapeutic use. Wound Repair Regen, 2016. 24(4): p. 613-29.
38. Gombozhapova, A., et al., Macrophage activation and polarization in post-infarction cardiac remodeling. J Biomed Sci, 2017. 24(1): p. 13.
39. Hu, Y., et al., Class A scavenger receptor attenuates myocardial infarction-induced cardiomyocyte necrosis through suppressing M1 macrophage subset polarization. Basic Res Cardiol, 2011. 106(6): p. 1311-28.
40. Ma, Y., et al., Matrix metalloproteinase-28 deletion exacerbates cardiac dysfunction and rupture after myocardial infarction in mice by inhibiting M2 macrophage activation. Circ Res, 2013. 112(4): p. 675-88.
41. Lee, C. W., et al., Macrophage heterogeneity of culprit coronary plaques in patients with acute myocardial infarction or stable angina. Am J Clin Pathol, 2013. 139(3): p. 317-22.
42. Yan, X., et al., Temporal dynamics of cardiac immune cell accumulation following acute myocardial infarction. J Mol Cell Cardiol, 2013. 62: p. 24-35.
43. Fernandez-Velasco, M., S. Gonzalez-Ramos, and L. Bosca, Involvement of monocytes/macrophages as key factors in the development and progression of cardiovascular diseases. Biochem J, 2014. 458(2): p. 187-93.
44. de Couto, G., et al., Macrophages mediate cardioprotective cellular postconditioning in acute myocardial infarction. J Clin Invest, 2015. 125(8): p. 3147-62.
45. Guiteras, R., M. Flaquer, and J. M. Cruzado, Macrophage in chronic kidney disease. Clin Kidney J, 2016. 9(6): p. 765-771.
46. Meng, X. M., et al., Macrophage Phenotype in Kidney Injury and Repair. Kidney Dis (Basel), 2015. 1(2): p. 138-46.
47. Yamamoto, S., et al., Atherosclerosis following renal injury is ameliorated by pioglitazone and losartan via macrophage phenotype. Atherosclerosis, 2015. 242(1): p. 56-64.
48. Li, C., et al., Enhanced M1 and Impaired M2 Macrophage Polarization and Reduced Mitochondrial Biogenesis via Inhibition of AMP Kinase in Chronic Kidney Disease. Cell Physiol Biochem, 2015. 36(1): p. 358-72.
49. Sun, Y. Y., et al., Macrophage Phenotype in Liver Injury and Repair. Scand J Immunol, 2017. 85(3): p. 166-174.
50. Gratchev, A., et al., Mphi1 and Mphi2 can be re-polarized by Th2 or Th1 cytokines, respectively, and respond to exogenous danger signals. Immunobiology, 2006. 211(6-8): p. 473-86.
51. Mills, C. D., M1 and M2 Macrophages: Oracles of Health and Disease. Crit Rev Immunol, 2012. 32(6): p. 463-88.
52. Mills, C. D. and K. Ley, M1 and M2 macrophages: the chicken and the egg of immunity. J Innate Immun, 2014. 6(6): p. 716-26.
53. Alsaid, H., et al., Non invasive imaging assessment of the biodistribution of GSK2849330, an ADCC and CDC optimized anti HER3 mAb, and its role in tumor macrophage recruitment in human tumor-bearing mice. PLoS One, 2017. 12(4): p. e0176075.
54. Josephs, D. H., et al., Anti-Folate Receptor-alpha IgE but not IgG Recruits Macrophages to Attack Tumors via TNFalpha/MCP-1 Signaling. Cancer Res, 2017. 77(5): p. 1127-1141.
55. Velmurugan, R., et al., Macrophage-Mediated Trogocytosis Leads to Death of Antibody-Opsonized Tumor Cells. Mol Cancer Ther, 2016. 15(8): p. 1879-89.
56. Gul, N. and M. van Egmond, Antibody-Dependent Phagocytosis of Tumor Cells by Macrophages: A Potent Effector Mechanism of Monoclonal Antibody Therapy of Cancer. Cancer Res, 2015. 75(23): p. 5008-13.
57. Church, A. K., et al., Anti-CD20 monoclonal antibody-dependent phagocytosis of chronic lymphocytic leukaemia cells by autologous macrophages. Clin Exp Immunol, 2016. 183(1): p. 90-101.
58. Shi, Y., et al., Trastuzumab triggers phagocytic killing of high HER2 cancer cells in vitro and in vivo by interaction with Fcgamma receptors on macrophages. J Immunol, 2015. 194(9): p. 4379-86.
59. Weiskopf, K. and I. L. Weissman, Macrophages are critical effectors of antibody therapies for cancer. MAbs, 2015. 7(2): p. 303-10.
60. Oflazoglu, E., et al., Macrophages contribute to the antitumor activity of the anti-CD30 antibody SGN-30. Blood, 2007. 110(13): p. 4370-2.
61. Osman, R., et al., Calreticulin Release at an Early Stage of Death Modulates the Clearance by Macrophages of Apoptotic Cells. Front Immunol, 2017. 8: p. 1034.
62. Feng, M., et al., Macrophages eat cancer cells using their own calreticulin as a guide: roles of TLR and Btk. Proc Natl Acad Sci USA, 2015. 112(7): p. 2145-50.
63. Chao, M. P., et al., Calreticulin is the dominant pro-phagocytic signal on multiple human cancers and is counterbalanced by CD47. Sci Transl Med, 2010. 2(63): p. 63ra94.
64. Murata, Y., et al., The CD47-SIRPalpha signalling system: its physiological roles and therapeutic application. J Biochem, 2014. 155(6): p. 335-44.
65. Roberts, D. D., S. Kaur, and D. R. Soto-Pantoja, Therapeutic targeting of the thrombospondin-1 receptor CD47 to treat liver cancer. J Cell Commun Signal, 2015. 9(1): p. 101-2.
66. Liu, J., et al., Pre-Clinical Development of a Humanized Anti-CD47 Antibody with Anti-Cancer Therapeutic Potential. PLoS One, 2015. 10(9): p. e0137345.
67. Weiskopf, K., et al., CD47-blocking immunotherapies stimulate macrophage-mediated destruction of small-cell lung cancer. J Clin Invest, 2016. 126(7): p. 2610-20.
68. Weiskopf, K., et al., Eradication of Canine Diffuse Large B-Cell Lymphoma in a Murine Xenograft Model with CD47 Blockade and Anti-CD20. Cancer Immunol Res, 2016. 4(12): p. 1072-1087.
69. Zeng, D., et al., A fully human anti-CD47 blocking antibody with therapeutic potential for cancer. Oncotarget, 2016. 7(50): p. 83040-83050.
70. Liljefors, M., et al., Influence of varying doses of granulocyte-macrophage colony-stimulating factor on pharmacokinetics and antibody-dependent cellular cytotoxicity. Cancer Immunol Immunother, 2008. 57(3): p. 379-88.
71. Tarr, P. E., Granulocyte-macrophage colony-stimulating factor and the immune system.

Med Oncol, 1996. 13(3): p. 133-40.
72. Ragnhammar, P., et al., Cytotoxicity of white blood cells activated by granulocyte-colony-stimulating factor, granulocyte/macrophage-colony-stimulating factor and macrophage-colony-stimulating factor against tumor cells in the presence of various monoclonal antibodies. Cancer Immunol Immunother, 1994. 39(4): p. 254-62.
73. Ragnhammar, P., Anti-tumoral effect of GM-CSF with or without cytokines and monoclonal antibodies in solid tumors. Med Oncol, 1996. 13(3): p. 167-76.
74. Lin, E. Y., et al., Colony-stimulating factor 1 promotes progression of mammary tumors to malignancy. J Exp Med, 2001. 193(6): p. 727-40.
75. Aharinejad, S., et al., Colony-stimulating factor-1 blockade by antisense oligonucleotides and small interfering RNAs suppresses growth of human mammary tumor xenografts in mice. Cancer Res, 2004. 64(15): p. 5378-84.
76. Lin, E. Y., et al., Macrophages regulate the angiogenic switch in a mouse model of breast cancer. Cancer Res, 2006. 66(23): p. 11238-46.
77. Lin, E. Y. and J. W. Pollard, Tumor-associated macrophages press the angiogenic switch in breast cancer. Cancer Res, 2007. 67(11): p. 5064-6.
78. Zhang, W. J., et al., Hypoxia-inducible factor-1 alpha Correlates with Tumor-Associated Macrophages Infiltration, Influences Survival of Gastric Cancer Patients. J Cancer, 2017. 8(10): p. 1818-1825.
79. Yuan, X., et al., Prognostic significance of tumor-associated macrophages in ovarian cancer: A meta-analysis. Gynecol Oncol, 2017. 147(1): p. 181-187.
80. Ma, C., et al., CD163-positive cancer cells are potentially associated with high malignant potential in clear cell renal cell carcinoma. Med Mol Morphol, 2017.
81. Shi, Y., et al., Tumour-associated macrophages secrete pleiotrophin to promote PTPRZ1 signalling in glioblastoma stem cells for tumour growth. Nat Commun, 2017. 8: p. 15080.
82. Zhao, X., et al., Prognostic significance of tumor-associated macrophages in breast cancer: a meta-analysis of the literature. Oncotarget, 2017. 8(18): p. 30576-30586.
83. Pearce, 0.M., et al., Inverse hormesis of cancer growth mediated by narrow ranges of tumor-directed antibodies. Proc Natl Acad Sci USA, 2014. 111(16): p. 5998-6003.
84. Pander, J., et al., Activation of tumor-promoting type 2 macrophages by EGFR-targeting antibody cetuximab. Clin Cancer Res, 2011. 17(17): p. 5668-73.
85. Clynes, R. A., et al., Inhibitory Fc receptors modulate in vivo cytotoxicity against tumor targets. Nat Med, 2000. 6(4): p. 443-6.
86. Pricop, L., et al., Differential modulation of stimulatory and inhibitory Fc gamma receptors on human monocytes by Th1 and Th2 cytokines. J Immunol, 2001. 166(1): p. 531-7.
87. Tridandapani, S., et al., Regulated expression and inhibitory function of Fcgamma RIM in human monocytic cells. J Biol Chem, 2002. 277(7): p. 5082-9.
88. Joshi, T., et al., Molecular analysis of expression and function of hFcgammaRllbl and b2 isoforms in myeloid cells. Mol Immunol, 2006. 43(7): p. 839-50.
89. Wijngaarden, S., et al., A shift in the balance of inhibitory and activating Fcgamma receptors on monocytes toward the inhibitory Fcgamma receptor lib is associated with prevention of monocyte activation in rheumatoid arthritis. Arthritis Rheum, 2004. 50(12): p. 3878-87.
90. Zumwalt, T. J., et al., Active secretion of CXCL10 and CCLS from colorectal cancer microenvironments associates with GranzymeB+CD8+T-cell infiltration. Oncotarget, 2015. 6(5): p. 2981-91.
91. Ochsenbein, A. F., Principles of tumor immunosurveillance and implications for immunotherapy. Cancer Gene Ther, 2002. 9(12): p. 1043-55.
92. Ochsenbein, A. F., et al., Roles of tumour localization, second signals and cross priming in cytotoxic T-cell induction. Nature, 2001. 411(6841): p. 1058-64.
93. Buhtoiarov, I. N., et al., CD40 ligation activates murine macrophages via an IFN-gamma-dependent mechanism resulting in tumor cell destruction in vitro. J Immunol, 2005. 174(10): p. 6013-22.
94. Egilmez, N. K., et al., Human CD4+ effector T cells mediate indirect interleukin-12- and interferon-gamma-dependent suppression of autologous HLA-negative lung tumor xenografts in severe combined immunodeficient mice. Cancer Res, 2002. 62(9): p. 2611-7.
95. Pace, J. L., et al., Recombinant mouse gamma interferon induces the priming step in macrophage activation for tumor cell killing. J Immunol, 1983. 130(5): p. 2011-3.
96. Heusinkveld, M., et al., M2 macrophages induced by prostaglandin E2 and IL-6 from cervical carcinoma are switched to activated M1 macrophages by CD4+Th1 cells. J Immunol, 2011. 187(3): p. 1157-65.
97. Hu, G. and S. Wang, Tumor-infiltrating CD45R0+Memory T Lymphocytes Predict Favorable Clinical Outcome in Solid Tumors. Sci Rep, 2017. 7(1): p. 10376.
98. Lohneis, P., et al., Cytotoxic tumour-infiltrating T lymphocytes influence outcome in resected pancreatic ductal adenocarcinoma. Eur J Cancer, 2017. 83: p. 290-301.
99. Liu, S., et al., Role of Cytotoxic Tumor-Infiltrating Lymphocytes in Predicting Outcomes in Metastatic HER2-Positive Breast Cancer: A Secondary Analysis of a Randomized Clinical Trial. JAMA Oncol, 2017: p. e172085.
100. Berntsson, J., et al., The clinical impact of tumour-infiltrating lymphocytes in colorectal cancer differs by anatomical subsite: A cohort study. Int J Cancer, 2017. 141(8): p. 1654-1666.
101. Xu, Y., et al., Higher Numbers of T-Bet+Tumor-Infiltrating Lymphocytes Associate with
Better Survival in Human Epithelial Ovarian Cancer. Cell Physiol Biochem, 2017. 41(2): p. 475-483.
102. Melief, S. M., et al., Long-term Survival and Clinical Benefit from Adoptive T-cell Transfer in Stage IV Melanoma Patients Is Determined by a Four-Parameter Tumor Immune Signature. Cancer Immunol Res, 2017. 5(2): p. 170-179.
103. Scurr, M. J., et al., Low-dose cyclophosphamide induces anti-tumor T-cell responses which associate with survival in metastatic colorectal cancer. Clin Cancer Res, 2017.
104. Wang, L., et al., Arsenic trioxide is an immune adjuvant in liver cancer treatment. Mol Immunol, 2017. 81: p. 118-126.
105. Ouyang, Z., et al., Regulatory T cells in the immunotherapy of melanoma. Tumour Biol, 2016. 37(1): p. 77-85.
106. Dimeloe, S., et al., Human regulatory T cells lack the cyclophosphamide-extruding transporter ABCB1 and are more susceptible to cyclophosphamide-induced apoptosis. Eur J Immunol, 2014. 44(12): p. 3614-20.
107. Camisaschi, C., et al., Effects of cyclophosphamide and IL-2 on regulatory CD4+T cell frequency and function in melanoma patients vaccinated with HLA-class I peptides: impact on the antigen-specific T cell response. Cancer Immunol Immunother, 2013. 62(5): p. 897-908.
108. Kan, S., et al., Suppressive effects of cyclophosphamide and gemcitabine on regulatory T-cell induction in vitro. Anticancer Res, 2012. 32(12): p. 5363-9.
109. Farsam, V., et al., Antitumor and immunomodulatory properties of artemether and its ability to reduce CD4+CD25+FoxP3+T reg cells in vivo. Int Immunopharmacol, 2011. 11(11): p. 1802-8.
110. Stockmann, C., et al., The impact of the immune system on tumor: angiogenesis and vascular remodeling. Front Oncol, 2014. 4: p. 69.
111. Hirst, D. G., J. Denekamp, and B. Hobson, Proliferation kinetics of endothelial and tumour cells in three mouse mammary carcinomas. Cell Tissue Kinet, 1982. 15(3): p. 251-61.
112. Denekamp, J. and B. Hobson, Endothelial-cell proliferation in experimental tumours. Br J Cancer, 1982. 46(5): p. 711-20.
113. Hobson, B. and J. Denekamp, Endothelial proliferation in tumours and normal tissues: continuous labelling studies. Br J Cancer, 1984. 49(4): p. 405-13.
114. Folkman, J., Tumor angiogenesis: therapeutic implications. N Engl J Med, 1971. 285(21): p. 1182-6.
115. Folkman, J., Fighting cancer by attacking its blood supply. Sci Am, 1996. 275(3): p. 150-4.
116. Ellis, L. M. and I. J. Fidler, Finding the tumor copycat. Therapy fails, patients don't. Nat Med, 2010. 16(9): p. 974-5.
117. Wagner, S. C., et al., Cancer anti-angiogenesis vaccines: Is the tumor vasculature antigenically unique? J Transl Med, 2015. 13: p. 340.
118. Wei, Y. Q., et al., Immunotherapy of tumors with xenogeneic endothelial cells as a vaccine. Nat Med, 2000. 6(10): p. 1160-6.
119. Zhang, W., J. N. Liu, and X. Y. Tan, Vaccination with xenogeneic tumor endothelial proteins isolated in situ inhibits tumor angiogenesis and spontaneous metastasis. Int J Cancer, 2009. 125(1): p. 124-32.
120. Luo, Y., et al., Immunotherapy of tumors with protein vaccine based on chicken homologous Tie-2. Clin Cancer Res, 2006. 12(6): p. 1813-9.
121. He, Q. M., et al., Inhibition of tumor growth with a vaccine based on xenogeneic homologous fibroblast growth factor receptor-1 in mice. J Biol Chem, 2003. 278(24): p. 21831-6.
122. Wagner, S. C., et al., Safety of targeting tumor endothelial cell antigens. J Transl Med, 2016. 14: p. 90.
123. Ichim, T. E., et al., Induction of tumor inhibitory anti-angiogenic response through immunization with interferon Gamma primed placental endothelial cells: ValloVax. J Transl Med, 2015. 13: p. 90.

1. A method of inducing an immune response to tumor associated blood vessels in a patient in need comprising the steps of: a) obtaining a pluripotent stem cell; b) modifying said pluripotent stem cell so as to induce an effect selected from the group consisting of: expression of an immunogenic molecule, loss of expression of an immunosuppressive molecule, and loss of expression of an immune suppressive signaling molecule; c) inducing differentiation of said pluripotent stem cell into endothelial cells under conditions which replicate the tumor microenvironment; d) obtaining said endothelial cells differentiated under conditions that replicate said tumor microenvironment and substantially isolating said cells in order to obtain a relatively homogeneous population of cells which resemble tumor endothelium associated cells; e) mitotically inactivating said cells; and f) administering said cells to a patient in need in a manner to stimulate an immune response.

2. The method of claim 1, wherein said tumor associated blood vessels possess expression of TEM-1.

3. The method of claim 1, wherein said tumor associated blood vessels possess expression of VEGF-receptor.

4. The method of claim 1, wherein said tumor associated blood vessels possess expression of nestin.

5. The method of claim 1, wherein said tumor associated blood vessels possess expression of TREM-1.

6. The method of claim 1, wherein said pluripotent stem cell is capable of forming a teratoma when placed in an immunodeficient mouse.

7. The method of claim 1, wherein said pluripotent stem cell is capable of forming ectoderm, mesoderm and endoderm tissue.

8. The method of claim 1, wherein said pluripotent stem cell is generated as an inducible pluripotent stem cell.

9. The method of claim 1, wherein said pluripotent stem cell is transfected with an immune stimulatory cytokine whose expression is either constitutive or inducible.

10. The method of claim 9, wherein said immune stimulatory cytokine is RANTES.

11. The method of claim 9, wherein said immune stimulatory cytokine is MIP-1 alpha.

12. The method of claim 9, wherein said immune stimulatory cytokine is MIP-1 beta.

13. The method of claim 9, wherein said immune stimulatory cytokine is interleukin-1.

14. The method of claim 1, wherein said pluripotent stem cell is gene edited to remove expression of IL-10 receptor.

15. The method of claim 1, wherein said pluripotent stem cell is gene edited to remove expression of IL-4 receptor.

16. The method of claim 1, wherein said endothelial cells are generated from said pluripotent cell by: a) culturing or maintaining a plurality of substantially undifferentiated pluripotent cells in a defined media comprising at least one growth factor; b) culturing the pluripotent cells in a defined media comprising an amount of BMP4 and VEGF sufficient to expand or promote differentiation in a plurality of the cells; and c) culturing the cells of (b) in a defined media comprising a composition selected from the group consisting of: (1) IL-3 and Flt3 ligand, (2) VEGF, FGF-2, and (3) an FGF-2 mimic, in combination with IGF sufficient to further expand or promote differentiation in a plurality of the cells; wherein a plurality of the pluripotent cells are differentiated into hematopoietic precursor cells or endothelial cells.

17. The method of claim 16, wherein the defined media of step (b) further comprises FGF-2 or an FGF-2 mimic.

18. The method of claim 16, wherein the defined media of step (c) comprises IL-3, Flt3 ligand, and GMCSF.

19. The method of claim 18, wherein the defined media of step (c) comprises IL-3, Flt3 ligand, and a compound selected from the group consisting of: IL-6, SCF, or TPO.

20. The method of claim 19, wherein the defined media of step (c) further comprises IL-6, SCF, and TPO.
